(19) United States
(12) Patent Application Publication
(10) Pub. No.: US 2011/0015624 A1
(43) Pub. Date: Jan. 20, 2011

(54) CRYOSURGICAL INSTRUMENT INSULATING SYSTEM

(75) Inventors: Didier Toubia, Ra'anana (IL); Alexander Levin, Binyamina (IL); Miron Kaganovich, Haifa (IL)

Correspondence Address: HESLIN ROTHENBERG FARLEY & MESITI PC, 5 COLUMBIA CIRCLE, ALBANY, NY, 12203, US

(73) Assignee: ICECURE MEDICAL LTD., Caesarea (IL)

(21) Appl. No.: 12/812,819

(22) PCT Filed: Jan. 15, 2009
(86) PCT No.: PCT/IL09/00062

§ 371 Date: Sep. 29, 2010

Related U.S. Application Data
(60) Provisional application No. 61/021,209, filed on Jan. 15, 2008.

(51) Int. Cl. A61B 18/02

(52) U.S. Cl. 606/23

(57) ABSTRACT

A device, system and method for controlling the temperature differential between the medium within the cryosurgical instrument and the external temperature of the shaft, apart from at the cryotip itself, uses a fluid medium supplied within an internal space between the shaft and an intermediate lumen positioned between the shaft and a central feeding lumen of the cryosurgical instrument. The temperature of the fluid medium is controlled to provide control of the temperature differential.

### FIELD OF THE INVENTION

The present invention relates to a system and method for insulating a cryosurgical instrument and in particular, to such a system and method for maintaining a controlled external temperature for at least a portion of the probe shaft.

### BACKGROUND OF THE INVENTION

Cryoprobes or catheters frequently experience problems of temperature control, particularly with regard to maintaining a temperature differential between the contents of the cryoprobe or catheter, which are very cold, and the outer shaft, which is desirably maintained at a higher temperature, outside of the cryotips themselves. The outer shaft is in contact with body tissues which may be damaged by excessively low temperatures of this shaft, as only the portion of the body tissues which are surrounding the cryotip should be frozen.

In addition, cryosurgical catheters must have a great deal of flexibility, especially when they are used for cardiac interventions. At the same time the closed distal end (cryotip) of such a probe or catheter must provide in many cases high specific freezing capacity at sufficiently low temperatures. Various attempted solutions to this problem have been provided with regard to thermal insulation of lateral non-operating walls of cryosurgical instruments.

For example, U.S. Pat. No. 3,971,383 proposes a cryogenic surgical instrument with a coaxial assembly of flexible lumens; the inner lumen is connected to a supply of cryogenic liquid, and the space between the outer wall of the inner lumen and the next lumen forms a return line for evaporated cryogenic liquid which is vented to the atmosphere. The space between the outermost one of the coaxial lumens and the intermediate lumen contains a gas, such as normal butane, serving for thermal insulation of the inner and intermediate lumens.

U.S. Pat. No. 5,573,532 describes a cryosurgical instrument, which comprises lumens of cryogenic fluid supply and return of cryogenic fluid vapors; these lumens are situated concentrically and the return lumen is sealed with a cryotip. Vacuum insulation of the return lumen is taught, which is very expensive and has low reliability. In addition, this vacuum insulation limits flexibility of the probe, especially when it has significant length and is used as a catheter.

U.S. Pat. No. 5,674,218 describes a cryosurgical instrument, a system and method of cryosurgery. According to this patent a cryogenic liquid (preferably, liquid nitrogen) is initially sub-cooled below its normal boiling point and then it is supplied into the open proximal end of the internal supply line. The outer lumen of the cryosurgical instrument is provided with active vacuum insulation.

U.S. Pat. No. 7,288,089 describes an enhanced method and device intended to treat atrial fibrillation or inhibit or reduce restenosis following angioplasty or stent placement. A balloon-tipped catheter is disposed in the area treated or opened through balloon angioplasty immediately following angioplasty. The balloon, which can have a dual balloon structure, may be delivered through a guiding catheter and over a guidewire already in place. A fluid such as a perfluorocarbon flows into the balloon to freeze the tissue adjacent the balloon, this cooling being associated with reduction of restenosis. A similar catheter may be used to reduce atrial fibrillation by inserting and inflating the balloon such that an exterior surface of the balloon contacts at least a partial circumference of the portion of the pulmonary vein adjacent the left atrium. In another embodiment, blood perfusion is performed simultaneously. In another embodiment, tissue contacted by the cryoablation catheter, which should not be ablated, is protected against damage by a separate heating step. However, this invention is limited to balloon catheters.

U.S. Pat. No. 7,273,479 describes methods and systems which are applied for cooling an object with a cryogen having a critical point defined by a critical-point pressure and a critical-point temperature. A pressure of the cryogen is raised above a pressure value determined to provide the cryogen at a reduced molar volume that prevents vapor lock. Thereafter, the cryogen is placed in thermal communication with the object to increase a temperature of the cryogen along a thermodynamic path that maintains the pressure greater than the critical-point pressure for a duration that the cryogen and object are in thermal communication.

U.S. Pat. No. 7,255,693 discloses a cryosurgical catheter which is heated in order to prevent its freezing within the lumen of an endoscope. The catheter is to be used with an endoscope to perform cryoablation on an internal tissue; e.g., the esophagus. Electric conductivity to produce heat employs an electrical conductive coating on the catheter. Also, disclosed is a fitting for use with a catheter comprising both a connection for receiving gas and an electrical connection.

U.S. Pat. No. 6,562,030 describes a cryocatheter which includes a catheter body defining a coolant flow path, a catheter tip exposed to the coolant flow path, and a heating element associated with the catheter tip. The heating element can be disposed entirely or partially within the catheter tip. Alternatively, the heating element can be external to the catheter tip. The heating element can include an electrically resistive element. However, it should be noted that in any case the heating element is an active electrical element.

US patent application No. 20070276360 discloses a cryosurgical catheter which is heated in order to prevent its freezing within the lumen of an endoscope. The catheter is to be used with an endoscope to perform cryoablation on an internal tissue; e.g., the esophagus. Electric conductivity to produce heat employs an electrical conductive coating on the catheter. Also, disclosed is a fitting for use with a catheter comprising both a connection for receiving gas and an electrical connection; again note that electrical power is required for the heating element.

In addition, U.S. Pat. Nos. 6,182,666, 6,095,149, 5,906,612, 5,899,897, 5,658,276 describe different versions of application of electrical heating elements for thermal insulation of untreated tissue.

U.S. Pat. Nos. 5,910,104 and 6,457,212 describe the application of thermo-insulating disposable sheaths, which are situated on shafts of cryosurgical instruments.

### SUMMARY OF THE INVENTION

The background art does not teach or suggest a simple and inexpensive mechanism for maintaining a controlled temperature differential between material inside a cryosurgical instrument and the external temperature of the shaft, away from the tip. The present invention overcomes these drawbacks of the background art, by providing a system and method for controlling the temperature differential between the material within the cryosurgical instrument and the external temperature of the shaft, apart from at the cryotip itself. The present invention uses a fluid within a jacket surrounding the shaft or between an intermediate lumen positioned coaxially with the central feeding lumen containing the cryogen and the outer shaft. The temperature of the fluid is controlled to maintain and/or to induce the above temperature differential. By “'fluid” it is understood that any gas, liquid or other material may optionally be used, alone or in combination. According to some embodiments of the present invention, a gas is preferred. According to other embodiments of the present invention, the cryosurgical instrument is preferably a cryocatheter which is optionally and more preferably flexible. According to other embodiments of the present invention, the cryosurgical instrument is preferably a cryoprobe which may optionally be non-flexible or less flexible.

A cryosurgical instrument and its accessory system are based on application of cryogen liquids with sufficiently low boiling temperatures at pressures in the interval from below one atmosphere through several atmospheres. On the other hand, these temperatures are sufficiently high for the use of gases with low thermal conductivity such as krypton as a dynamic or static thermal insulator, when the operating pressure of these gases is maintained at such a level that these gases are in their superheated condition, so that they cannot condense at the operating temperatures of the cryogen.

It should be noted that mixtures of some gases can be implemented as the cryogen and/or as thermo-insulating gas.

However, as a preferred embodiment, the present invention preferably comprises liquid CF4 (Freon R 14) as a working cryogen and krypton as a thermo-insulating gas. According to another embodiment, liquid nitrogen is used as a freezing agent and warm gaseous nitrogen as an active thermo-insulating medium. Preferably a cryosurgical instrument according to the present invention comprises an external elongated shaft; a central feeding lumen positioned in the external elongated shaft for receiving a cryogen; a cryotip, which is joined sealingly with the distal edge of the external elongated shaft; an intermediate lumen positioned coaxially between the central feeding lumen of the external elongated shaft and joined sealingly with the external elongated shaft by its distal and proximal flanges, which features a fluid medium for controlling the temperature differential between the external shaft and the central feeding lumen.

In addition, the proximal end of the central feeding lumen is preferably terminated by an inlet connection for receiving cryogen, while the proximal section of the elongated external shaft, which seals the internal space between the central feeding lumen and this shaft, is preferably provided with an outlet connection for permitting the cryogen gas to be exhausted out. A proximal section of the elongated external shaft, which bounds the internal space with the intermediate lumen, is provided as well with an inlet connection. In another embodiment, this last inlet connection can serve alternatively as an inlet and outlet connection.

The cryosurgical instrument preferably operates as follows. The working cryogen (liquid nitrogen, CF4 or another cryogen with a sufficiently low boiling temperature in the desired range of pressures) is supplied in the form of separated pulses with sufficiently low on-off ratio into the central feeding lumen via its proximal inlet connection. By “sufficiently low on-off ratio” it is meant that the timing of the pulses is such so as to cool the tip sufficiently, preferably while maintaining the presence of continuously boiling cryogen at the tip. The liquid component of the working cryogen accumulates in the internal space of the cryotip and is preferably constantly boiling with cooling of the wall of the cryotip and freezing of the surrounding tissue.

At the same time a heating and/or thermo-insulating medium is preferably supplied into the space between the elongated external shaft and the intermediate lumen via its inlet-outlet connection. In such a way the pressure in the space between the elongated external shaft and the intermediate lumen is elevated.

Thereafter the pressurized gas between the elongated external shaft and the intermediate lumen is purged through this inlet-outlet connection.

After terminating the cryogen pulse, the pressurized heating and thermo-insulating medium again enters the internal space between the elongated external shaft and the intermediate lumen.

In another embodiment, the thermo-insulating medium is optionally introduced into the space between the elongated external shaft and the intermediate lumen periodically. In yet another embodiment, the distal flange of the intermediate lumen is provided with through openings for passage of the thermo-insulating medium. In such a way, the thermo-insulating medium flows via the gap between the elongated external shaft and the intermediate lumen and mixes with the evaporated cryogen exhausted from the cryotip. This allows the temperature of the exhausted gas to be elevated and, in such a way, to decrease the cooling effect of this exhausted gas on the elongated external shaft. The thermo-insulating medium may optionally be provided to space between the intermediate lumen and the elongated external shaft during the intervals between cryogen pulses or alternatively (and optionally) may be provided continuously if the pressure of the thermo-insulating medium upon entering the gap between the elongated external shaft and the intermediate lumen exceeds the pressure of the exhausted cryogen at the distal section of the gap between the central feeding lumen and the intermediate lumen.

### BRIEF DESCRIPTION OF THE DRAWINGS

FIG. 1a is an axial cross-section of an assembled cryoprobe according to the present invention in some embodiments;

FIG. 1 b is an axial cross-section of the male unit of the above cryoprobe; FIG. 1 c is an axial cross-section of the female unit of the cryoprobe of FIG. 1 a; and

FIG. 2 is an axial cross-section of another embodiment of a cryoprobe according to the present invention.

### DESCRIPTION OF PREFERRED EMBODIMENTS

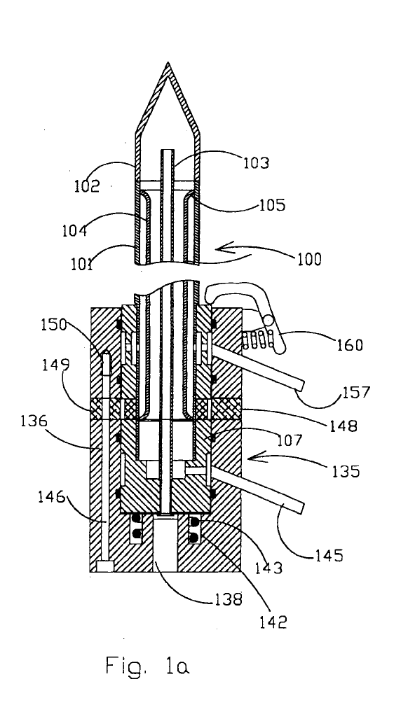

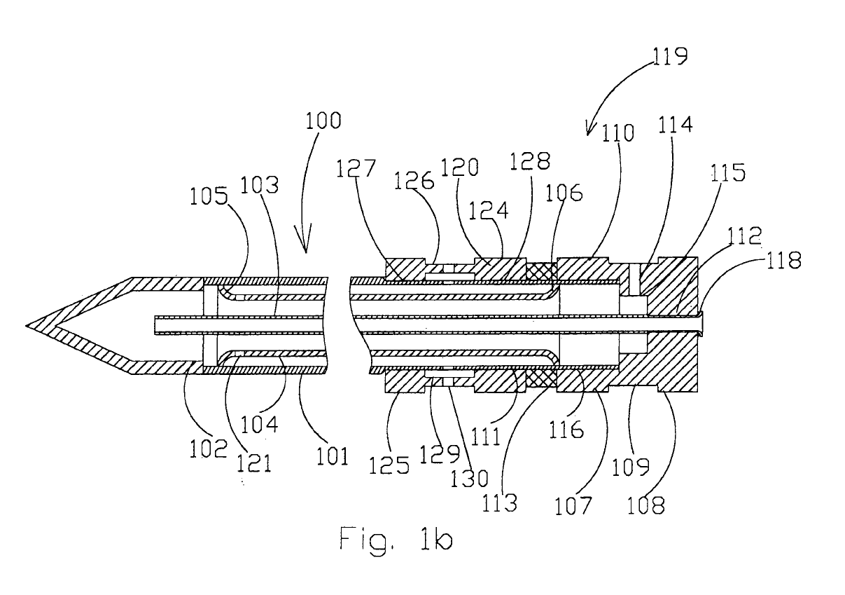

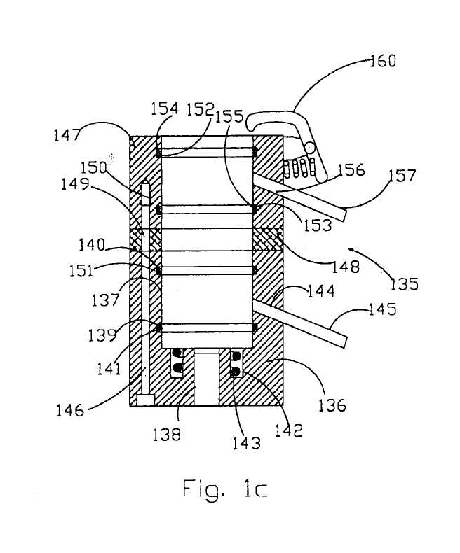

FIG. 1 a, FIG. 1 b and FIG. 1 c show an axial cross-section of a cryoprobe according to some embodiments of the present invention with an intermediate lumen for receiving a temperature controlling fluid, preferably a heating and insulating gas, and a quick coupling construction for easy assembly. This embodiment of cryoprobe 100 comprises the elongated external shaft 101, which terminates at its distal edge with cryotip 102. A central feeding pipe 103 is situated in shaft 101. Preferably, the proximal end of the central feeding pipe 103 protrudes from the proximal end of shaft 101. The proximal sections of the elongated external shaft 101 and the central feeding pipe 103 serve for installation of a male unit 119 for quick coupling. The extreme proximal section of the shaft 101 is preferably somewhat radially projected to prevent movement past a certain point with regard to male unit 119.

Thermal insulation of the elongated external shaft 101 is ensured by an intermediate tube 104 with two flanged ends 105 and 106, wherein the outer diameter of the formed flanges 105 and 106 conforms to the internal diameter of the shaft. Friction between the internal surface of the elongated external shaft 101 and flanged ends 105 and 106 ensures stable positioning of the intermediate tube 104 relative to the elongated external shaft 101. The proximal section 111 of shaft 101 is preferably provided with at least one and preferably a plurality of openings 121, which allow fluid communication with the internal space between the intermediate lumen 104 and the elongated external shaft 101 for receiving the fluid temperature controlling material. The male unit 119 of the quick coupling, which is installed on the proximal sections of the elongated external shaft 101 and the central feeding pipe 103, preferably comprises a first bushing 107; the outer and internal surfaces of this first bushing 107 are preferably stepped.

The outer surface of the first bushing 107 preferably comprises proximal and distal cylindrical sections 108 and 110 and a middle section 109; the proximal and distal sections 108 and 110 have the same diameter, while the diameter of the middle section 109 is somewhat smaller.

The inner surface of the first bushing 107 is preferably also stepped: it preferably has distal, middle and proximal sections 116, 115 and 112 with progressively reduced diameters.

The first bushing 107 is installed on the proximal sections of shaft 101 and the central feeding pipe 103 such that the distal section of the inner surface of the bushing 107 is fitted tightly on the proximal section of the shaft 101, while the proximal inner surface 112 of bushing 107 is fitted slidingly on the proximal section of the central feeding pipe 103. After positioning the first bushing 107 on the proximal section of the elongated external shaft 101, the proximal edge of the central feeding pipe 103 is preferably flanged with application of a deformable o-ring 118, more preferably constructed from a cryogenically stable polymer, for sealing the gap between the proximal sections of the internal surface of the first bushing 107 and the central feeding pipe 103. A first channel 114 communicates between the internal and external spaces of inner middle section 115 and outer middle section 109 of the first bushing 107.

A second bushing 120 is preferably installed on the longitudinally turned section 111 of the elongated external shaft 101 distally to the first bushing 107 and spaced from this first bushing 107 by a thermo-insulating ring 113. The outer surface of the second bushing 120 preferably comprises proximal and distal cylindrical sections 124 and 125 and a middle section 126; the proximal and distal sections 124 and 125 have the same diameter, while the diameter of the middle section 126 is somewhat smaller.

In a similar manner, the inner surface of the second bushing 120 preferably comprises proximal and distal cylindrical sections 128 and 127 and a middle section 129; the proximal and distal sections 128 and 127 have the same diameter, while diameter of the middle section 129 is somewhat larger.

Preferably a plurality of openings 130 in the middle section 126 provide fluid communication from the internal space between the intermediate lumen 104 and the elongated external shaft 101 to the external space which is external to cryoprobe 100. A female unit 135 of the quick coupling mechanism preferably comprises a proximal housing 136, with the cylindrical inner cavity 137, wherein the diameter of the cylindrical inner cavity 137 conforms to the outer diameters of the distal and proximal sections 110 and 108 of the first bushing 107 of male unit 119.

An opening 138 in the proximal face plane of the inner cavity serves for installation of an inlet connection supplying the cryogen into cryoprobe 100 (this inlet connection is not shown). It should be noted that the tolerance of the space between the first bushing 107 and the cylindrical inner cavity 137 permits the bushing 107 of cryoprobe 100 to be slidingly inserted into the housing 136 of the female unit 135. The cylindrical inner cavity 137 is preferably provided with a plurality of annular grooves 151 and 139, which serve for installation of corresponding polymer o-rings 140 and 141; these polymer o-rings 140 and 141 ensure sealing of the middle section 126 of the first bushing 107 of cryoprobe 100.

In addition, the inner surface of the face plane of the proximal housing 136 is preferably provided with an annular groove 142, and a helical spring 143, which is partially situated in annular groove 142. In such a way, in the process of coupling, the male unit 119 of the coupling pair is spring-actuated by this helical spring 143, to maintain male unit 119 in tight coupling to female unit 135.

Preferably a channel 144 with an outlet connection 145 installed on the outer end of channel 144 communicates between the middle section 109 of the first bushing 107 and the outside space of the proximal housing 136. The proximal housing 136 comprises an opening 146, with the axis situated in parallel to the axis of the cylindrical inner cavity 137; this opening 146 serves for installation of a joining screw, which is not shown.

In addition, the female unit 135 preferably comprises a distal bushing 147, which is separated from the proximal housing 136 by a thermo-insulating ring 148. The thermo-insulating ring 148 and the distal bushing 147 are preferably provided with an opening 149 and a blind hole 150 with threading 151 for installation of the aforementioned assembling screw (not shown), to close and lock male unit 119 and female unit 135.

The cylindrical inner surface of the distal bushing 147 is provided with two annular grooves 152 and 153, which serve for installation of two polymer o-rings 154 and 155; these polymer o-rings 154 and 155 ensure sealing of the middle section 126 of the second bushing 120 of cryoprobe 100.

A through channel 156 is provided with an inlet-outlet connection 157 for receiving the temperature controlling fluid material: this inlet-outlet connection 157 is installed on the outer end of the through channel 156 for connecting between the middle section 126 of the second bushing 120 and the inner surface of the distal bushing 147, and the outside space of the distal bushing 147.

In such a way, the distal bushing 147 may be used to supply a heating and thermo-insulating medium into the space between the elongated external shaft 101 and the intermediate lumen 104 through the inlet-outlet connection 157.

A spring-actuated ratchet 160 is installed on the outer surface of the distal bushing 147 permitting the male unit 119 and the female unit 135 to be coupled and uncoupled.

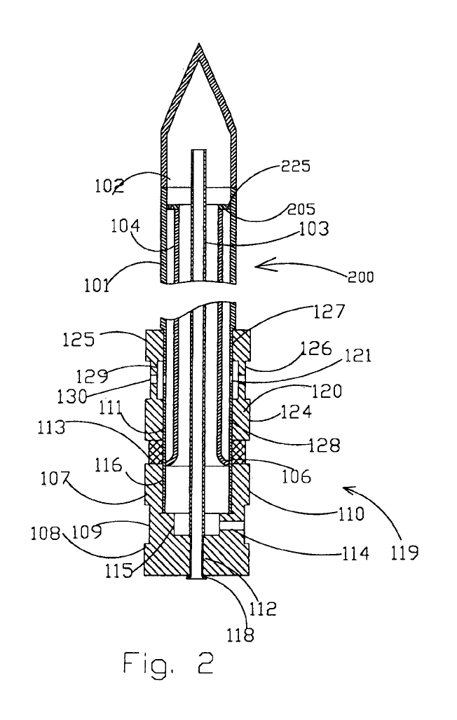

FIG. 2 shows an axial cross-section of another embodiment of a cryoprobe with a plurality of openings in the distal flange of its intermediate lumen for passage of the thermo-insulating medium.

The embodiment of cryoprobe 200 comprises the elongated external shaft 101, which ends at its distal edge with cryotip 102. Numbers which are identical to FIG. 1 have the same or identical function unless otherwise specified.

Thermal insulation of the elongated external shaft 101 is ensured by an intermediate tube 104 with two flanged ends 205 and 106, wherein the outer diameter of the formed flanges 205 and 106 conforms to the internal diameter of the shaft. Friction between the internal surface of the elongated external shaft 101 and flange 106 ensures stable positioning of the intermediate tube 104 within the elongated external shaft 101.

The distal flange 205 preferably features a plurality of openings 225, which ensures passage of a thermo-insulating gaseous medium and its mixture with the gaseous cryogen to be exhausted from cryotip 202. The medium preferably enters at openings 121 so that the fluid medium travels one way through intermediate tube 104. This avoids the need for charging and purging intermediate tube 104, as for the embodiment of FIG. 1.

Persons skilled in the art will appreciate that the present invention is not limited to what has been particularly shown and described hereinabove. Rather the scope of the present invention is defined by the appended claims and includes both combinations and sub combinations of the various features described hereinabove as well as variations and modifications thereof, which would occur to persons skilled in the art upon reading the foregoing description.

1. A cryosurgical instrument, comprising: a central feeding tube for receiving a cryogen, said central feeding tube terminating at or before a cryotip; an external shaft for containing said central feeding tube and terminating at said cryotip; and an intermediate tube between said central feeding tube and said external shaft, said intermediate tube terminating before said cryotip, wherein a space between said intermediate tube and said external shaft receives a fluid material for controlling a temperature differential between said external shaft and said intermediate tube, and wherein said space is at least partially sealed at opposite ends of said intermediate tube.

2. The instrument of claim 1, wherein said fluid material comprises a gaseous medium.

3. The instrument of claim 2, wherein said cryogen is supplied into said cryosurgical instrument in the form of separated pulses and said gaseous medium is supplied into said space between said external shaft and the intermediate tube during time intervals between said pulses.

4. The instrument of claim 3, wherein said gaseous medium after passage through said space between said external shaft and said intermediate tube is mixed with evaporated cryogen.

5. The instrument of claim 2, wherein said gaseous medium is in superheated vapor condition at its operating pressure and the temperature of said evaporated cryogen.

6. The instrument of claim 2, wherein said cryogen comprises liquid CF4 and wherein said gaseous medium comprises gaseous krypton.

7. The instrument of claim 2, wherein said space is fully sealed after at least one cycle of charging and purging said gaseous medium.

8. The instrument of claim 1, wherein said central tube further comprises a proximal inlet connection for receiving said cryogen, said intermediate tube further comprises an outlet connection for exhausting evaporated cryogen, said intermediate tube is sealed with an internal surface of said external shaft by distal and proximal flanges, the instrument further comprising: an inlet connection installed on said external shaft and communicating with said space between said intermediate tube and said external shaft for receiving said fluid material.

9. The instrument of claim 8, further comprising an outlet connection installed on said external shaft and communicating with said space between said intermediate tube and said external shaft for exhausting said fluid material.

10. The instrument of claim 9, wherein said inlet and said outlet connections are combined to a single inlet-outlet connection.

11. The instrument of claim 8, wherein said distal flange of the intermediate tube is provided with a plurality of openings.

12. The instrument of claim 2, wherein said gaseous medium is supplied continuously or intermittently to said space, such that said external shaft further comprises an inlet connection for receiving said gaseous medium to said space and an outlet connection for exhausting said gaseous medium from said space.

13. The instrument of claim 1, wherein said cryosurgical instrument comprises a flexible cryocatheter.

14. The instrument of claim 1, wherein said cryosurgical instrument comprises a rigid cryoprobe.

15. A method of dynamic temperature control of an external shaft of a cryosurgical instrument according to claim 2, said instrument featuring a channel in said external shaft for removal of evaporated cryogen by said intermediate tube, said method comprising: supplying a gaseous medium into said space between said intermediate tube and said elongated shaft; said gaseous medium being in superheated vapor condition at its operating pressure and the temperature of said evaporated cryogen.

16. The method of claim 15, further comprising supplying said cryogen into said cryosurgical instrument in the form of separated pulses, such that said gaseous medium is supplied into said space between said external shaft and the intermediate tube during time intervals between said pulses.

17. The method of claim 15, further comprising mixing said gaseous medium after its passage via said space between said external shaft and said intermediate tube with the evaporated cryogen.

18. The method of claim 15, wherein said cryogen comprises liquid CF4 and wherein said gaseous medium comprises gaseous krypton.
